(19) United States
(12) Patent Application Publication
(10) Pub. No.: US 2021/0137931 A1
(43) Pub. Date: May 13, 2021

(54) SELECTING PATIENTS FOR THERAPY WITH ADENOSINE SIGNALING INHIBITORS

(71) Applicant: ASTRAZENECA AB, SODERTALJE (SE)

(72) Inventors: Bolan LINGHU, Wilmington, DE (US); Melinda MERCHANT, Wilmington, DE (US); Kris SACHSENMEIER, Wilmington, DE (US); Mingchao XIE, I, Wilmington, DE (US)

(21) Appl. No.: 17/049,739

(22) PCT Filed: Apr. 23, 2019
(86) PCT No.: PCT/EP2019/060312

§ 371 Date: Oct. 22, 2020

Related U.S. Application Data
(60) Provisional application No. 62/661,930, filed on Apr. 24, 2018.

(51) Int. Cl. A61K 31/53; C07K 16/28; C12Q 1/6886

(52) U.S. Cl. CPC A61K 31/53; C12Q 2600/158; C12Q 1/6886; C07K 16/2896

(57) ABSTRACT

Described herein are measures of the relative expression levels, within a given sample from a subject, of an ACPP transmembrane splice variant to the expression level of one or more ACPP non-transmembrane splice variant(s). The measures, designated ρ, show correlation with clinical outcome for the subject. In some cases, values of ρ exceeding a predetermined cutoff can be associated with poorer outcomes. Methods of determining ρ and assigning the predetermined cutoff value are described. Methods of treating cancer are also described.

Immune-checkpoint inhibitors hold great potential as cancer therapeutics. Nevertheless, clinical benefits from immune-checkpoint inhibition have been modest. One potential explanation for the modest benefits is that tumors use nonoverlapping immunosuppressive mechanisms to facilitate immune escape.

Some cancers are typically considered unresponsive to immune checkpoint inhibitors; prostate cancer is one such cancer. One reason for this lack of response could be the presence of an immunosuppressive tumor microenvironment within tumors. Extracellular adenosine can suppress tumor infiltrating immune cells through a net negative impact of signaling through adenosine receptors, including the A2a receptor (A2aR). The primary source of extracellular adenosine in tumors is believed to be extracellular ATP, which is metabolized to AMP by the ectonucleotidase CD39, and then converted from AMP to adenosine by the ectonucleotidase CD73. CD73 is anchored to the cell membrane through a glycosylphosphatidylinositol (GPI) linkage. Production of adenosine by CD73 has been shown to regulate adenosine receptor engagement in many tissues, indicating that adenosine functions such as cytoprotection, cell growth, angiogenesis and immunosuppression, and also plays a role in tumorigenesis.

CD73 expression on tumor cells has been reported in several types of cancer, including colorectal cancer, pancreatic cancer, bladder cancer, leukemia, lymphoma, glioma, glioblastoma, melanoma, ovarian cancer, thyroid cancer, esophageal cancer, prostate cancer, and breast cancer. Elevated CD73 expression has also been associated with tumor invasiveness, metastasis, and reduced patient survival time.

In addition to CD73, another enzyme produces extracellular adenosine in the prostate (including prostate tumors): prostatic acid phosphatase (PAP, gene name ACPP). Because it catalyzes the same conversion of AMP to adenosine as CD73, PAP could function as an ortholog to CD73 within tumors.

### SUMMARY

In one aspect, a method for treating an elevated adenosine cancer in a subject includes diagnosing the subject with an elevated adenosine cancer when, in a sample from the subject, a measure ρ of the relative expression levels of an ACPP TM variant to the expression level of one or more ACPP non-TM variant(s) exceeds a predetermined cutoff value; and administering an effective amount of an adenosine signaling inhibitor to the diagnosed subject.

The cancer can be prostate cancer, lung cancer, bladder cancer, or other cancer. The predetermined cutoff value of ρ is the median, mean, top quartile, top quintile, top decile, or other statistical measure, of ρ in a selected group of reference samples.

ρ can be the log 2 of the ratio of the expression level of ACPP variant 2 to the expression level of ACPP variant 1, 3, 4, 5, 6, 7, 8, 9, or the total expression level of a combination thereof. ρ can be the log 2 of the ratio of the expression level of ACPP variant 2 to the expression level of ACPP variant 1. ρ can be the log 2 of the ratio of the expression level of ACPP variant 2 to the expression level of ACPP variant 3. ρ can be the log 2 of the ratio of the expression level of ACPP variant 2 to the total expression level of ACPP variants 1 and 3. ρ can be the log 2 of the ratio of the expression level of ACPP variant 2 to the total expression level of ACPP variants 1, 3, 4, 5, 6, 7, 8, and 9.

The adenosine signaling inhibitor can include a CD39 inhibitor, a CD73 inhibitor, a PAP inhibitor, an adenosine receptor antagonist, or a combination thereof. The CD73 inhibitor can be MEDI9447 or AB680. The adenosine receptor antagonist can be an antagonist of A2aR and/or A2bR. The adenosine receptor antagonist can be AZD4635, CPI-444, PBF-509, PBF-1129, or preladenant.

The adenosine signaling inhibitor can include a CD73 inhibitor and an adenosine receptor antagonist. The CD73 inhibitor can be MEDI9447 and the adenosine receptor antagonist is AZD4635.

The sample can be a tumor sample, a circulating tumor DNA (ctDNA) sample, a plasma RNA sample, or an exosome sample.

In one aspect, an adenosine signaling inhibitor can be used in the treatment of an elevated adenosine cancer in a subject in need thereof, wherein: in a sample from the subject, a measure ρ of the relative expression levels of an ACPP TM variant to the expression level of one or more ACPP non-TM variant(s) exceeds a predetermined cutoff value.

Other features, objects, and advantages will be apparent from the description and drawings, and from the claims.

### BRIEF DESCRIPTION OF THE DRAWINGS

FIG. 1A, FIG. 1B, and FIG. 1C shows relative expression levels of NT5E (the gene encoding CD73) and ACPP (the gene encoding PAP) across a variety of tumor types. Dark circles, ACPP; open circles, NT5E.

FIG. 2 plots the log 2 of the ratio of the expression levels of TM variant to non-TM variant of ACPP in normal prostate (left) and in primary prostate tumors (right).

FIG. 3A shows a Cox regression analysis to assess the association between the log 2 of the ratio of TM variant to non-TM variant and the clinical outcome with tumor stage and diagnosis age as covariates. Hazard ratio and p-values are shown.

FIG. 3B is a Kaplan-Meier estimator plot illustrating the survival-rate difference between high-ρ patients (black) compared to low-ρ patients (grey). The cutoff between high-ρ and low-ρ was defined based on the median log 2 of the ratio of TM-variant to non-TM variant across all prostate primary tumor samples.

FIG. 3C compares ρ with different primary Gleason histology patterns using box plots, ranging from of Gleason scores of 2 to 5, with 2 representing least severe status and 5 representing most severe status. The Kruskal-Wallis test was performed to compare ρ across different categories with the p-value calculated to show the significant difference.

FIG. 3D compares ρ across tumors with different tumor stages using box plots, ranging from stages T2a to T4, with T2a representing least severe status and T4 representing the most severe status. The median of each group is labeled. The Kruskal-Wallis test was performed across different categories with the p-value calculated to show significant difference.

FIG. 4 plots the log 2 of the ratio of the expression levels of TM variant to non-TM variant of ACPP in normal prostate (left), in primary prostate tumors (center), and in metastases (right).

### DETAILED DESCRIPTION

PAP levels were shown to be elevated in the blood of patients with prostate cancer over 50 years ago. PAP activity was found to be elevated in patients with metastases and was widely used as a surrogate marker for prostate cancer. The uses of PAP is no longer standard in screening and clinical management since serum prostate specific antigen (PSA) was validated as a prognostic marker for patients with prostate cancer.

More recently, a broad survey of The Cancer Genome Atlas (TCGA; a public database containing genomic, transcriptomic, clinical, and proteomic data across a wide range of cancer indications) showed that ACPP was approximately 300 fold more highly-expressed in prostate tumors than NT5E (the gene encoding CD73). Furthermore, ACPP was much more highly expressed than NT5E in all tumors surveyed (FIG. 1A, FIG. 1B, FIG. 1C,). These observations suggested that the relatively high levels of adenosine-producing enzyme PAP might give prostate tumors a distinct survival advantage due to immunosuppressive adenosine within the tumor microenvironment, even though the use of blood PAP levels are no longer considered a reliable marker of prostate cancer.

A total of nine mRNA splice variants of ACPP RNA have been identified. Three of these are major forms that are commonly found, while the other six minor forms represent a small portion of the total ACPP mRNA. Only one splice variant of ACPP, one of the major forms, includes a region that encodes a transmembrane (TM) domain. Thus, PAP is found in different isoforms, including membrane-bound (TM-PAP) and secreted (non-TM-PAP) isoforms. As discussed in greater detail below, it is now surprisingly found that a measure of the relative expression levels of TM-PAP to non-TM-PAP expression is predictive of clinical outcome.

With TM-PAP being a membrane-bound protein, it can be expected that local extracellular adenosine concentrations in proximity to cells with higher TM-PAP expression may be greater than in other environments where TM-PAP expression is low. Without intending to be bound to any mechanism, it is proposed that greater expression of TM-PAP (relative to non-TM-PAP) in some tumors can lead to locally increased adenosine concentrations, thereby leading to immunosuppression in the tumor microenvironment. Immunosuppression in the tumor would in turn favor tumor survival, leading to poorer clinical outcomes.

Without intending to be bound to any mechanism, the survival of tumors expressing relatively more TM-PAP may be more reliant on adenosine-mediated immunosuppression than tumors exhibiting expressing relatively less TM-PAP. Tumors expressing relatively more TM-PAP may therefore be more sensitive to inhibition of adenosine signaling (and subsequent decrease in immune suppression). Thus, subjects with tumors expressing relatively more TM-PAP may show enhanced response to an adenosine signaling inhibitor relative to subjects whose tumors express relatively less TM-PAP.

As used herein, the term “ACPP” refers to the human gene encoding prostatic acid phosphatase, i.e., the gene cataloged at ensembl database entry ENSG00000014257 (uswest.ensembl.org/Homo_sapiens/Gene/Summary?db=core;g=ENSG00000014257;r=3:13231 7367-132368298, genome version of GRCh38.p10).

As used herein, the term “variant 1” refers to ACPP splice variant 1, the splice variant cataloged at ensembl database entry ENSG00000014257, transcript ID ENST00000336375.9 (ACPP-201). As used herein, the term “isoform 1” refers to PAP isoform 1, the PAP protein isoform encoded by variant 1. Variant 1 is one of the commonly found ACPP splice variants.

As used herein, the term “variant 2” refers to ACPP splice variant 2, the splice variant cataloged at ensembl database entry ENSG00000014257, transcript ID ENST00000351273.11 (ACPP-202). As used herein, the term “isoform 2” refers to PAP isoform 2, i.e., the PAP protein isoform encoded by variant 2. Variant 2, uniquely among the ACPP splice variants, encodes a transmembrane domain. Variant 2 is one of the commonly found ACPP splice variants.

As used herein, the term “TM variant” refers to the ACPP splice variant which encodes a PAP protein isoform which includes a transmembrane domain. In other words, the term “TM variant” is synonymous with “variant 2”.

As used herein, the term “TM-PAP” refers to the PAP protein isoform which includes a transmembrane domain. In other words, the term “TM-PAP” is synonymous with “isoform 2”.

As used herein, the term “variant 3” refers to ACPP splice variant 3, the splice variant cataloged at ensembl database entry ENSG00000014257, transcript ID ENST00000475741.5 (ACPP-203). As used herein, the term “isoform 3” refers to PAP isoform 3, i.e., the PAP protein isoform encoded by variant 3. Variant 3 is one of the commonly found ACPP splice variants.

As used herein, the term “variant 4” refers to ACPP splice variant 4, the splice variant cataloged at ensembl database entry ENSG00000014257, transcript ID ENST00000483689.1 (ACPP-204).

As used herein, the term “variant 5” refers to ACPP splice variant 5, the splice variant cataloged at ensembl database entry ENSG00000014257, transcript ID ENST00000489084.5 (ACPP-205).

As used herein, the term “variant 6” refers to ACPP splice variant 6, the splice variant cataloged at ensembl database entry ENSG00000014257, transcript ID ENST00000493235.5 (ACPP-206).

As used herein, the term “variant 7” refers to ACPP splice variant 7, the splice variant cataloged at ensembl database entry ENSG00000014257, transcript ID ENST00000495911.5 (ACPP-207).

As used herein, the term “variant 8” refers to ACPP splice variant 8, the splice variant cataloged at ensembl database entry ENSG00000014257, transcript ID ENST00000507647.1 (ACPP-208).

As used herein, the term “variant 9” refers to ACPP splice variant 9, the splice variant cataloged at ensembl database entry ENSG00000014257, transcript ID ENST00000512463.1 (ACPP-209).

As used herein, the term “non-TM variant(s)” refers to one or more ACPP splice variants which encode a PAP protein isoform that does not include a transmembrane domain. In other words, “non-TM variant(s)” refers to one or more of variants 1, 3, 4, 5, 6, 7, 8, and 9.

As used herein, the term “non-TM-PAP” refers collectively to protein isoforms 1, 3, 4, 5, 6, 7, 8, and 9. In other words, “non-TM-PAP” refers collectively to all of the non-membrane-bound (i.e., soluble) PAP isoforms.

As used herein, the term “PAP,” if not otherwise specified, refers collectively to the all of the protein isoforms encoded by ACPP, i.e., “PAP” refers collectively to isoforms 1-9.

As used herein, the symbol “ρ” refers to a measure, for a given sample, of the relative expression levels of an ACPP TM variant to the expression level of one or more ACPP non-TM variant(s). In some embodiments, ρ can be the log 2 of the ratio of expression values, for a given sample, of the expression level of an ACPP TM variant to the expression level of one or more ACPP non-TM variant(s).

As used herein, the term “elevated adenosine cancer” refers to a cancer in which adenosine levels are elevated in the tumor microenvironment relative to surrounding non-tumor tissue. An elevated adenosine cancer can, in some embodiments, be an adenosine receptor antagonist-sensitive cancer. As used herein, “an adenosine receptor antagonist-sensitive cancer” refers to a cancer that responds to treatment with an adenosine receptor antagonist (whether alone or in combination with another treatment). The adenosine receptor antagonist can be an antagonist of one or more of the A1R, A2aR, A2bR, and A3R adenosine receptors.

In some embodiments, a subject can be diagnosed with an elevated adenosine cancer if the value of p, in a sample from the subject, exceeds a predetermined cutoff value. For a given tumor type, the predetermined cutoff value can be assigned by first calculating the value of ρ for a large number of reference samples (e.g., at least 25, at least 50, at least 100, or more). The reference samples can be, for example, from different patients; and/or the same patients at different time points. The predetermined cutoff value can then be assigned after analysis of the values of ρ of the reference samples. The predetermined cutoff value can be assigned as the median, mean, top quartile, top quintile, top decile, or other statistical measure of the values of ρ of the reference samples. In some embodiments, the cutoff value is the median value of ρ of the reference samples. In some embodiments, the cutoff value can depend on the specific distributions of ρ of the reference samples.

The cutoff value can be different for different tumor types. In this context, a tumor type refers not only to the type or location of the cancer (e.g., prostate cancer or lung cancer), but can also refer to a narrower set of tumors, characterized by features such as tumor stage, mutation status of one or more genes, biomarker status, sensitivity to a given therapy, microsatellite instability, and others. Thus, even within a given type of cancer, sub-populations may be identified for which a different value of ρ is selected as the cutoff value. As one illustrative example, castration resistant prostate cancer and castration sensitive prostate cancer can be considered different tumor types, as that term is used herein.

As used herein, the term “adenosine signaling inhibitor” refers to a compound (including without limitation small molecules and biologics) which interacts with one or more components of the adenosine signaling pathway in a manner capable of decreasing adenosine signaling. Thus, adenosine signaling inhibitors include, without limitation, compounds that inhibit the production of adenosine and compounds that antagonize one or more adenosine receptors. Thus, adenosine signaling inhibitors include compounds that inhibit enzyme(s) that directly or indirectly produce adenosine including, for example, CD39, CD73, and PAP. Examples of CD39 inhibitors include IPH52 and POM-1. Examples of CD73 inhibitors include MEDI9447 and AB680. Adenosine signaling inhibitors also include compounds that antagonize one or more adenosine receptors (including, for example, AIR, A2aR, A2bR and A3R). Examples of adenosine receptor antagonists include without limitation AZD4635 (chemical name: 6-(2-chloro-6-methylpyridin-4-yl)-5-(4-fluorophenyl)-1,2,4-triazin-3-amine), CPI-444, PBF-509, PBF-1129, and preladenant.

Antibodies and antibody-like compounds (e.g., monoclonal antibodies, antibody fragments, and the like) that bind to CD39, CD73, PAP, or an adenosine receptor can also be adenosine signaling inhibitors. Adenosine signaling inhibitors can also include compounds that inhibit downstream components of the adenosine signaling pathway.

Administration of one or more adenosine signaling inhibitors to a subject diagnosed with an elevated adenosine cancer can promote a positive therapeutic response with respect to the elevated adenosine cancer. As used herein, the term “positive therapeutic response,” encompasses a reduction or inhibition of the progression and/or duration of cancer, the reduction or amelioration of the severity of cancer, and/or the amelioration of one or more symptoms thereof. For example, a reduction or inhibition of the progression and/or duration of cancer can be characterized as a complete response. The term “complete response” refers to an absence of clinically detectable disease with normalization of any previously abnormal test results. Alternatively, an improvement in the disease can be categorized as being a partial response.

In some illustrative examples, a positive therapeutic response includes one, two or three or more of the following results: (1) a stabilization, reduction or elimination of the cancer cell population; (2) a stabilization or reduction in cancer growth; (3) an impairment in the formation of cancer; (4) eradication, removal, or control of primary, regional and/or metastatic cancer; (5) an increase in anti-cancer immune response; (6) a reduction in mortality; (7) an increase in disease-free, relapse-free, progression-free, and/or overall survival, duration, or rate; (8) an increase in the response rate, the durability of response, or number of patients who respond or are in remission; (9) a decrease in hospitalization rate, (10) a decrease in hospitalization lengths, (11) the size of the cancer is maintained and does not increase or increases by less than 10%, preferably less than 5%, preferably less than 4%, preferably less than 2%, (12) an increase in the number of patients in remission, and (13) a decrease in the number or intensity of adjuvant therapies (e.g., chemotherapy or hormonal therapy) that would otherwise be required to treat the cancer.

In one aspect, a method for treating cancer (e.g., an elevated adenosine cancer) in a subject can include: diagnosing the subject with an elevated adenosine cancer when, in a sample from the subject, a measure ρ of the relative expression levels of an ACPP TM variant to the expression level of one or more ACPP non-TM variant(s) exceeds a predetermined cutoff value; and administering an effective amount of an adenosine signaling inhibitor to the diagnosed subject.

In one aspect, a method for treating cancer (e.g., an elevated adenosine cancer) in a subject can include: measuring, in a sample from the subject, a measure ρ of the relative expression levels of an ACPP TM variant to the expression level of one or more ACPP non-TM variant(s), wherein the measured value of ρ exceeds a predetermined cutoff value; and administering an effective amount of an adenosine signaling inhibitor to the diagnosed subject.

In one aspect, a method for treating cancer (e.g., an elevated adenosine cancer) in a subject can include: obtaining a sample from the subject; measuring, in the sample from the subject, a measure ρ of the relative expression levels of an ACPP TM variant to the expression level of one or more ACPP non-TM variant(s), wherein the measured value of ρ exceeds a predetermined cutoff value; and administering an effective amount of an adenosine signaling inhibitor to the diagnosed subject.

In one aspect, a method for treating cancer (e.g., an elevated adenosine cancer) in a subject can include: identifying a subject having a value of ρ that exceeds a predetermined cutoff value, wherein ρ is a measure of the relative expression levels of an ACPP TM variant to the expression level of one or more ACPP non-TM variant(s); and administering an effective amount of an adenosine signaling inhibitor to the diagnosed subject.

In one aspect, a method of identifying a subject having a cancer suited to treatment with an adenosine signaling inhibitor can include: determining that a measure ρ of the relative expression levels of an ACPP TM variant to the expression level of one or more ACPP non-TM variant(s) exceeds a predetermined cutoff value.

In one aspect, a method of identifying an elevated adenosine cancer can include: determining a measure ρ of the relative expression levels of an ACPP TM variant to the expression level of one or more ACPP non-TM variant(s) in a sample from the subject; determining whether ρ exceeds a predetermined cutoff value.

In one aspect, a method of treating cancer (e.g., an elevated adenosine cancer) can include: determining a measure ρ of the relative expression levels of an ACPP TM variant to the expression level of one or more ACPP non-TM variant(s) in a sample from the subject; determining whether ρ exceeds a predetermined cutoff value; and administering an effective amount of an adenosine signaling inhibitor to the diagnosed subject.

In one aspect, an adenosine signaling inhibitor can be for use in the treatment of cancer (e.g., an elevated adenosine cancer) in a subject in need thereof, wherein: in a sample from the subject, a measure ρ of the relative expression levels of an ACPP TM variant to the expression level of one or more ACPP non-TM variant(s) exceeds a predetermined cutoff value.

In one aspect, a method of predicting a subject's response to a cancer treatment can include: comparing the relative expression levels of an ACPP TM variant to the expression level of one or more ACPP non-TM variant(s) in a sample from the subject; and determining if a measure ρ of the relative expression levels exceeds a predetermined cutoff value.

In one aspect, a method of diminishing adenosine-mediated immunosuppression in a tumor of a subject can include: determining whether, in a sample from the subject, a measure ρ of the relative expression levels of an ACPP TM variant to the expression level of one or more ACPP non-TM variant(s) exceeds a predetermined cutoff value; and administering an effective amount of an adenosine signaling inhibitor to the subject if ρ exceeds the predetermined cutoff value.

In some embodiments, the elevated adenosine cancer can be prostate cancer, lung cancer, bladder cancer, or other cancer. In some embodiments, the elevated adenosine cancer can be prostate cancer.

In some embodiments, the sample is a tumor sample (e.g., a biopsy sample), a circulating tumor DNA (ctDNA) sample, a plasma RNA sample, an exosome sample, or other blood-derived sample.

The expression levels of different ACPP variants in a sample can be measured by any method that can quantify mRNA levels in a sample and distinguish between the TM variant and one or more non-TM variant(s). In some embodiments, it may not be necessary to distinguish non-TM variants from other non-TM variants. Suitable methods for measuring expression levels of different ACPP variants include, but are not limited to, RNAseq, qPCR, or platform-specific assays such as microarrays or nanostring analysis.

In some embodiments, ρ is the log 2 of the ratio of the expression level of ACPP variant 2 to the expression level of ACPP variant 1, 3, 4, 5, 6, 7, 8, 9, or the total expression level of a combination thereof.

In some embodiments, ρ is the log 2 of the ratio of the expression level of ACPP variant 2 to the expression level of ACPP variant 1.

In some embodiments, ρ is the log 2 of the ratio of the expression level of ACPP variant 2 to the expression level of ACPP variant 3.

In some embodiments, ρ is the log 2 of the ratio of the expression level of ACPP variant 2 to the expression level of ACPP variant 4.

In some embodiments, ρ is the log 2 of the ratio of the expression level of ACPP variant 2 to the expression level of ACPP variant 5.

In some embodiments, ρ is the log 2 of the ratio of the expression level of ACPP variant 2 to the expression level of ACPP variant 6.

In some embodiments, ρ is the log 2 of the ratio of the expression level of ACPP variant 2 to the expression level of ACPP variant 7.

In some embodiments, ρ is the log 2 of the ratio of the expression level of ACPP variant 2 to the expression level of ACPP variant 8.

In some embodiments, ρ is the log 2 of the ratio of the expression level of ACPP variant 2 to the expression level of ACPP variant 9.

In some embodiments, ρ is the log 2 of the ratio of the expression level of ACPP variant 2 to the total expression level of ACPP variants 1 and 3.

In some embodiments, ρ is the log 2 of the ratio of the expression level of ACPP variant 2 to the total expression level of ACPP variants 1, 3, 4, 5, 6, 7, 8 and 9.

The adenosine signaling inhibitor can include a CD39 inhibitor, CD73 inhibitor, a PAP inhibitor, an adenosine receptor antagonist, or a combination thereof.

In some embodiments, the adenosine signaling inhibitor is a CD39 inhibitor. The CD39 inhibitor can be IPH52 or POM-1.

In some embodiments, the adenosine signaling inhibitor is a CD73 inhibitor. The CD73 inhibitor can be MEDI9447 or AB680.

In some embodiments, the adenosine signaling inhibitor is an adenosine receptor antagonist. In some embodiments, the adenosine receptor is an antagonist of A2aR and/or A2bR. The adenosine signaling antagonist can be selective for A2aR or for A2bR. The adenosine receptor antagonist can be AZD4635, CPI-444, PBF-509, PBF-1129, or preladenant.

In some embodiments, the adenosine signaling inhibitor can be a combination of a CD73 inhibitor and an adenosine receptor antagonist. In some embodiments, the CD73 inhibitor is MEDI9447 and the adenosine receptor antagonist is AZD4635.

In some embodiments, the cancer is prostate cancer.

In some embodiments, the cancer is prostate cancer, and ρ is the log 2 of the ratio of the expression level of ACPP variant 2 to the expression level of ACPP variant 1.

In some embodiments, the cancer is prostate cancer, and ρ is the log 2 of the ratio of the expression level of ACPP variant 2 to the expression level of ACPP variant 3.

In some embodiments, the cancer is prostate cancer, and ρ is the log 2 of the ratio of the expression level of ACPP variant 2 to the total expression level of ACPP variants 1 and 3.

In some embodiments, the cancer is prostate cancer, and ρ is the log 2 of the ratio of the expression level of ACPP variant 2 to the total expression level of ACPP variants 1, 3, 4, 5, 6, 7, 8, and 9.

In some embodiments, the cancer is prostate cancer, ρ is the log 2 of the ratio of the expression level of ACPP variant 2 to the expression level of ACPP variant 1, and the predetermined cutoff value is the median value of ρ from a large number of reference samples.

In some embodiments, the cancer is prostate cancer, ρ is the log 2 of the ratio of the expression level of ACPP variant 2 to the expression level of ACPP variant 3, and the predetermined cutoff value is the median value of ρ from a large number of reference samples.

In some embodiments, the cancer is prostate cancer, ρ is the log 2 of the ratio of the expression level of ACPP variant 2 to the total expression level of ACPP variants 1 and 3, and the predetermined cutoff value is the median value of ρ from a large number of reference samples.

### EXAMPLES

### Example 1

In studying the splice variants of ACPP, it was noted that only a single variant (variant 2) encoded a transmembrane domain which anchors the enzyme to the cell surface. Other variants encoded isoforms lacking this transmembrane domain. As these other non-TM isoforms are not anchored, they are not expected to be localized within a tumor microenvironment. Since ACPP is highly-expressed in both normal prostate and tumors, the relative expression levels of the ACPP variant were compared across both normal and tumor tissue. Although the TM-PAP-encoding variant was typically expressed at lower levels than the non-TM variants, the intra-sample log 2 of the ratio of TM-PAP to non-TM variants (i.e., ρ) was observed to be significantly higher in tumor samples than in normal prostate samples (FIG. 2).

In FIG. 2, the prostate cancer cohort was from TCGA (cancergenome.nih.gov). The mRNA expression measurements from RNAseq were downloaded from Omicsoft OncoLand (www.omicsoft.com). The clinical annotations of the samples such as sample types, tumor stages, clinical outcomes, etc., were downloaded from cBioportal (www.cbioportal.org). The log 2 of the ratio of measured ACPP variant 2 mRNA expression to measured ACPP variant 1 mRNA expression (an illustrative ρ measurement) was compared between 501 prostate tumor samples and 52 matched prostate normal samples by a t-test. The results indicated ρ was significantly higher in the prostate tumor samples than in the prostate normal samples (p-value <0.0001).

### Example 2

Further study of ρ in TCGA samples showed the association of ρ with known clinical outcomes. Similar associations were observed with covariates such as tumor stages (defined by American Joint Committee on Cancer ranging from T1 stage to T4 stage) and diagnosis ages, on clinical outcome (FIG. 3A). In particular, prostate cancer patients with high ρ show reduced disease-free survival rates, where the cutoff between “high” and “low” values of ρ was defined as the median ρ (FIG. 3B). Additionally, higher ρ is shown to be associated with more advanced disease progression measured by tumor stages defined by American Joint Committee on Cancer (FIG. 3C) and higher Gleason-pattern pathological scores (FIG. 3D), indicating greater risks and higher mortality.

For FIGS. 3A-3D, as above, the cohort was from TCGA prostate cancer patients; the mRNA expression measurement from RNAseq were downloaded from the Omicsoft OncoLand (www.omicsoft.com); the clinical annotations of the samples such as tumor stages, clinical outcomes, et al., were downloaded from cBioportal (www.cbioportal.org).

In FIG. 3A, a forest plot showed the Cox regression analysis of the effect of different variables on the clinical outcome measured by disease-free survival rate using the TCGA cohort of prostate cancer patients. The predictor variables include TMRatio, tumor stages, and diagnosis ages. TMRatio is an illustrative ρ measurement, which is defined here as the log 2 of the ratio of ACPP variant 2 mRNA expression to ACPP variant 1 mRNA expression from RNAseq; the TMRatio “high” category and the TMRatio “low” category are further defined using median ρ as the cutoff. Tumor stages defined as T2 or T3 (the two stages with large sample sizes) defined by American Joint Committee on Cancer with T3 representing more advanced disease stage than T2. The hazard ratios of the three variables on the disease-free survival rate were shown together with the corresponding p-values, which indicated that high TMRatio is associated with reduced disease-free survival rate (hazard ratio of 1.5, p-value of 0.093).

FIG. 3B shows the Kaplan Meier plot of disease-free survival rate in the same TCGA prostate cancer cohort as in FIG. 3A, the patients were grouped into the TMRatio “high” group and the TMRatio “low” group as described above. The risk table at bottom shows the total number of subjects at risk for both groups at each different time point. The plot indicates that high TMRatio is associated with reduced disease-free survival rate.

FIG. 3C shows the TMRatio boxplots in the TCGA prostate cancer cohort across different groups based on primary Gleason pattern scores, which are used as typical metrics for prostate-tumor pathology grading, with a higher score indicating a more advanced disease with greater risks and higher mortality. The Kruskal-Wallis test across the groups indicates that higher TMRatio is associated with higher primary Gleason pattern scores (p-value=1.2e-5).

FIG. 3D shows the TMRatio boxplots in the TCGA prostate cancer cohort across different groups based on tumor stages defined by American Joint Committee on Cancer ranging from T2b stage to T4 stages, with a higher stage indicating a more advanced disease with greater risks and higher mortality. The Kruskal-Wallis test across the groups indicate that higher TMRatio is associated with more advanced cancer stages (p-value=4.5e-7).

### Example 3

A survey of normal prostate as well as primary and metastatic prostate tumor tissue gene expression microarray data was made from a publication by Taylor B S et al. “Integrative genomic profiling of human prostate cancer.” Cancer Cell. 18, 11-22 (2010), which is incorporated by reference in its entirety. Probes were identified which specifically recognized the long TM variant of ACPP and other probes which recognized the short non-TM variants. By comparing the ratio of long and short ACPP variants, it was demonstrated that the expression ratio of long to short transcripts was significantly higher in metastatic tumors when compared with either normal tissue or primary tumor (FIG. 4). Consistent with TCGA data (Examples 1 and 2 above), the ratio in primary prostate tumor tissue was also higher than normal prostate tissue.

Other embodiments are within the scope of the following claims.

What is claimed is:

1. A method for treating an elevated adenosine cancer in a subject, comprising:
diagnosing the subject with an elevated adenosine cancer when, in a sample from the subject, a measure ρ of the relative expression levels of an ACPP TM variant to the expression level of one or more ACPP non-TM variant(s) exceeds a predetermined cutoff value; and
administering an effective amount of an adenosine signaling inhibitor to the diagnosed subject.

2. The method of claim 1, wherein the cancer is prostate cancer, lung cancer, bladder cancer, or other cancer.

3. The method of any one of claims 1 to 2, wherein the predetermined cutoff value of ρ is the median, mean, top quartile, top quintile, top decile, or other statistical measure, of ρ in a selected group of reference samples.

4. The method of any one of claims 1 to 3, wherein ρ is the log 2 of the ratio of the expression level of ACPP variant 2 to the expression level of ACPP variant 1, 3, 4, 5, 6, 7, 8, 9, or the total expression level of a combination thereof.

5. The method of any one of claims 1 to 3, wherein ρ is the log 2 of the ratio of the expression level of ACPP variant 2 to the expression level of ACPP variant 1.

6. The method of any one of claims 1 to 3, wherein ρ is the log 2 of the ratio of the expression level of ACPP variant 2 to the expression level of ACPP variant 3.

7. The method of any one of claims 1 to 3, wherein ρ is the log 2 of the ratio of the expression level of ACPP variant 2 to the total expression level of ACPP variants 1 and 3.

8. The method of any one of claims 1 to 3, wherein ρ is the log 2 of the ratio of the expression level of ACPP variant 2 to the total expression level of ACPP variants 1, 3, 4, 5, 6, 7, 8, and 9.

9. The method of any one of claims 1 to 8, wherein the adenosine signaling inhibitor includes a CD39 inhibitor, a CD73 inhibitor, a PAP inhibitor, an adenosine receptor antagonist, or a combination thereof.

10. The method of claim 9, wherein the CD73 inhibitor is MEDI9447 or AB680.

11. The method of any one of claims 9 to 10, wherein the adenosine receptor antagonist is an antagonist of A2aR and/or A2bR.

12. The method of any one of claims 9 to 11, wherein the adenosine receptor antagonist is AZD4635, CPI-444, PBF-509, PBF-1129, or preladenant.

13. The method of any one of claims 9 to 13, wherein the adenosine signaling inhibitor includes a CD73 inhibitor and an adenosine receptor antagonist.

14. The method of claim 13, wherein the CD73 inhibitor is MEDI9447 and the adenosine receptor antagonist is AZD4635.

15. The method of any one of claims 1 to 14, wherein the sample is a tumor sample, a circulating tumor DNA (ctDNA) sample, a plasma RNA sample, or an exosome sample.

16. An adenosine signaling inhibitor for use in the treatment of an elevated adenosine cancer in a subject in need thereof, wherein: in a sample from the subject, a measure ρ of the relative expression levels of an ACPP TM variant to the expression level of one or more ACPP non-TM variant(s) exceeds a predetermined cutoff value.
